(19) United States
(12) Patent Application Publication
(10) Pub. No.: US 2012/0059226 A1
(43) Pub. Date: Mar. 8, 2012

(54) SURGICAL RETRACTORS WITH ILLUMINATION

(76) Inventor: David K. Funt, Woodmere, NY (US)

(21) Appl. No.: 13/223,857

(22) Filed: Sep. 1, 2011

Related U.S. Application Data
(60) Provisional application No. 61/379,108, filed on Sep. 1, 2010.

(51) Int. Cl. A61B 1/06; A61B 1/32

(52) U.S. Cl. 600/213; 600/245

(57) ABSTRACT

A surgical retractor has a plurality of lights affixed to a blade thereof, that can illuminate a wound of a patient during operation. The lights can be on a distal end of the blade and/or an interior or exterior surface of the blade, so that the user can control the lighting on the retractor to suit the particular needs of a particular application. The lights can be powered by a battery compartment within the handle, allowing for untethered operation of the retractor.

### CROSS-REFERENCE TO RELATED APPLICATIONS

The present application claims priority to U.S. Provisional Patent No. 61/379108, filed on Sep. 1, 2010.

### BACKGROUND OF THE DISCLOSURE

1. Field of the Disclosure

The present disclosure relates to retractors used in surgical applications. In particular, the present disclosure relates to surgical retractors having light sources affixed thereon.

2. Discussion of the Related Art

Retractors are used in surgical applications to separate tissues from one another, or to hold back underlying organs in order to create an optical cavity for the surgeon. Currently, the area in the vicinity of the retractor is illuminated using halogen, xenon, or light-emitting diode (LED) light sources that are located remotely in the operating room, away from the surgical site. A flexible fiber optic cable is used to transmit the light from the source to the surgical site (wound). The light from the fiber optic cable is then transmitted to a fiber optic rod shielded by surgical steel, and welded to the retractor. The light is in this fashion delivered to the distal portion of the surgical retractor and into the wound.

This arrangement is undesirable for several reasons. The fiber optic is usually bulky and heavy, with a thick profile, and restricts the mobility of the retractor. Also, the tethered cord for the light source limits the mobility of the user, as it restricts movement around the surgical table and must remain sterile at all times. Since the fiber optics are made of glass, upon repeated exposures to the autoclave sterilization process, the fibers fracture, causing a drop in optical efficiency over time. In addition, movement of the cable as it is used also causes fracture of the glass fibers, reducing the amount of light delivered over time. Also, with fiber optics, the lighting arrangements are very limited. Fiber optic retractors in use today deliver light to the distal tip of the retractor only (i.e., spot lighting). They are not able to deliver flood lighting of a surgical cavity. In addition, they do not afford the surgeon the ability to switch from flood to spot lighting as needed. Lastly, the need to purchase a fiber optic light cord, a fiber optic retractor, and a halogen, xenon, or LED light source make the surgical illumination system unnecessarily costly. Xenon light sources, for example, are known to be particularly expensive.

Accordingly, there is a need to address these disadvantages.

### SUMMARY OF THE DISCLOSURE

The present disclosure addresses the problems with current retractors by providing a retractor that has small lights connected to the retractor itself, for example at the tip and/or on an exterior and/or interior surface of a blade of the retractor. These lights can either be powered by a battery source that is in the retractor handle, or there can be a power supply connection in the handle to a low voltage electrical cord that connects to a power supply.

Thus, in one embodiment, the present disclosure provides a surgical retractor comprising a handle having a battery compartment therein, a blade connected to the handle, at least one light permanently affixed to the blade. The light is in electrical communication with the battery compartment. The surgical retractor can further comprise a switch on the handle that is in electrical communicaton with the battery compartment a plurality of lights, wherein the switch is operable between four positions, to place the surgical retractor in one of four modes of operation.

In another embodiment, the present disclosure provides a surgical retractor comprising a handle having a port for connecting to an external source of power, a blade connected to the handle, wherein the blade comprises a distal end, a connection end connected to the handle, wherein the distal end is on an opposite side of the blade from the connection end, an exterior surface that runs along a length of the blade, and an interior surface that runs along a length of the blade on an opposite side of the blade from the exterior surface, and a plurality of light-emitting diodes permanently affixed to the blade.

### BRIEF DESCRIPTION OF THE DRAWINGS

FIG. 1 shows an embodiment of the retractor of the present disclosure.

### DETAILED DESCRIPTION OF THE DISCLOSURE

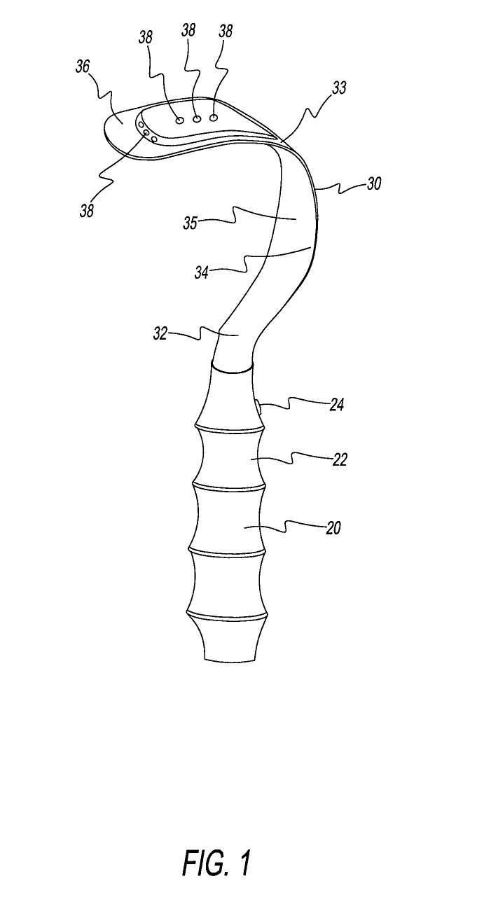

Referring to FIG. 1, retractor 10 is shown, and has handle 20 and blade 30. Blade 30 has first or handle connection end 32, body 34, and second or distal end 36, the latter of which can have a plurality of lights 38 thereon. Lights 38 can be used to illuminate a target area, such as the inside of the body cavity of a patient during operation. Lights 38 can be powered by a battery compartment 22 in handle 20, and be in electrical communication with battery compartment 22 with appropriate circuitry that runs from second end 36 through body 34, and out first end 32 into handle 20. Alternatively, lights 38 can be powered by an external source, which would be placed in electrical communication with lights 38 through a power connection on handle 20.

In the shown embodiment, lights 38 are LEDs. However, the present disclosure contemplates other possible lights, provided that they can withstand autoclave processes, are small enough to be mounted or connected to the blade of the retractor, and do not give off a significant amount of heat.

Thus, retractor 10 solves the above-described problems of current devices. It provides a slim and lightweight profile, since bulky and heavy fiber optics are not needed, which enhances the maneuverability for the user. Previously, it was not thought possible to have such a retractor design, since compact light sources were not powerful, inexpensive, or small enough mount to a retractor in the way they are in the present disclosure.

The number and arrangement of lights 38 will vary depending on the application. The present disclosure contemplates that lights 38 are arranged in such a way that they can provide spot lighting, by being located at or near the tip of the retractor blade (for example, those at second end 32 in FIG. 1). In addition, lights 38 will be able to provide flood lighting, by being located along the length of blade 30, along an exterior surface 33 and/or interior surface 35 of the blade (for example, the cluster of three lights 38 along exterior surface 33 of blade 30 in FIG. 1). This is another significant advantage of the retractor of the present disclosure. In the embodiment shown in FIG. 1, there are 3-5 lights 38 at the tip of second end 36 to provide spot lighting, and three additional lights 38 along exterior surface 33 to provide flood lighting. The present disclosure contemplates alternative arrangements of lights as well.

As previously discussed, no current devices have the ability to provide spot and flood lighting simultaneously, or even alternatively. In the retractor of the present disclosure, lights providing spot lighting and lights providing flood lighting can not only be on the same retractor, but can be connected to one switch 24 or to different switches 24, so that the user can alternate between them easily. The switch or switches 24 to control lights 38 can be on handle 20.

Handle 20 can be detachable from blade 30. First end 32 of blade 30 can be modified so that handle 20 can be removably connected to blade 30. This allows the user to swap out different blades. In addition, different handles can be used, for example one that uses an external power source, or one that has a battery compartment therein, as described above. In one embodiment, first end 32 has threads thereon, so that it can screw on to handle 20. Other methods of connecting blade 30 to handle 20 are contemplated, such as with a bayonet or friction fit connection. When handle 20 has a battery compartment therein, the batteries used can be disposable, or rechargeable. Handle 20 also be disposable, and come in a sterilized pouch, so that that the handle is one-time use, and would be universal in design able to fit any blade with the proposed product line.

Handle 20, blade 30, and lights 38 can be made from any material that can withstand an autoclave, to at least two hundred fifty-six degrees Fahrenheit, or that can be immersed in a disinfecting solution such as Cidex®. Such materials include, but are not limited to, surgical steel, ceramic and titanium. High impact temperature resistant plastics would be used in the disposable handles.

The shown embodiment illustrates a curved blade 30. The present disclosure, however, contemplates other blade designs that can have lights 38 placed thereon, for example a right angled blade where first end 32 and second end 36 would be straight, and at right angles to one another. Blade design could mimic those that are presently familiar to surgeons.

While the instant disclosure has been described with reference to one or more particular embodiments, it will be understood by those skilled in the art that various changes may be made and equivalents may be substituted for elements thereof without departing from the scope thereof. In addition, many modifications may be made to adapt a particular situation or material to the teachings of the disclosure without departing from the scope thereof. Therefore, it is intended that the disclosure not be limited to the particular embodiment(s) disclosed as the best mode contemplated for carrying out this disclosure.

What is claimed is:

1. A surgical retractor, comprising:
a handle having a battery compartment therein;
a blade connected to said handle; and
at least one light permanently affixed to said blade, wherein said light is in electrical communication with said battery compartment.

2. The surgical retractor of claim 1, wherein said at least one light is a light-emitting diode.

3. The surgical retractor of claim 1, wherein said blade comprises:
a distal end;
a connection end connected to said handle, wherein distal end is on an opposite side of said blade from said connection end;
an exterior surface that runs along a length of the blade; and
an interior surface that runs along a length of the blade on an opposite side of said blade from said exterior surface.

4. The surgical retractor of claim 3, wherein said at least one light is located at said distal end of said blade, to provide illumination in a direction away from said distal end.

5. The surgical retractor of claim 3, wherein said at least one light is located on said exterior surface, to provide illumination in a direction away from said exterior surface.

6. The surgical retractor of claim 3, wherein said at least one light is located on said interior surface, to provide illumination in a direction away from said interior surface.

7. The surgical retractor of claim 3, wherein said at least one light is a plurality of lights,
wherein at least one light is located at said distal end, and
wherein at least one light is located on at least one of said interior and said exterior surface.

8. The surgical retractor of claim 7, further comprising a switch on said handle that is in electrical communicaton with said battery compartment and said plurality of lights, wherein said switch is operable between four positions, to place the surgical retractor in one of the following modes of operation:
a power off mode, in which all of said plurality of lights are turned off;
a spot lighting mode, in which only said lights located at said distal end are turned on;
a flood lighting mode, in which only said lights on said interior or exterior surface are turned on; and
a combination lighting mode, in which at least one of said at least one lights at said distal end and at least one of said at least one lights on either said interior surface or said exterior surface are turned on.

9. The surgical retractor of claim 1, wherein said blade is removably connected to said handle.

10. A surgical retractor, comprising:
a handle having a port for connecting to an external source of power;
a blade connected to said handle, wherein said blade comprises a distal end, a connection end connected to said handle, wherein said distal end is on an opposite side of said blade from said connection end, an exterior surface that runs along a length of the blade, and an interior surface that runs along a length of the blade on an opposite side of said blade from said exterior surface; and
a plurality of light-emitting diodes permanently affixed to said blade.

11. The surgical retractor of claim 10, wherein at least one of said light-emitting diodes is permanently connected to said distal end and at least one of said light-emitting diodes is connected to at least one of said interior surface and said exterior surface.
